(19) United States
(12) Patent Application Publication
(10) Pub. No.: US 2021/0361235 A1
(43) Pub. Date: Nov. 25, 2021

(54) ELECTROENCEPHALOGRAM ELECTRODE CAP

(71) Applicant: WUHAN GREENTEK PTY. LTD, Wuhan, Hubei (CN)

(72) Inventors: Mingzhe Li, Wuhan, Hubei (CN); Yvonne Yanwen Duan, Wuhan, Hubei (CN); Detao Yang, Wuhan, Hubei (CN); Gongze Bai, Wuhan, Hubei (CN)

(21) Appl. No.: 16/624,962

(22) PCT Filed: Apr. 28, 2019
(86) PCT No.: PCT/CN2019/084775

§ 371 Date: Dec. 20, 2019

(51) Int. Cl. A61B 5/00; A61B 5/291; A61B 5/266

(52) U.S. Cl. CPC A61B 5/6803; A61B 5/291; A61B 2562/227; A61B 2562/046; A61B 5/266

(57) ABSTRACT

The present disclosure provides an electroencephalogram electrode cap, including: a stretchable covering body; an electrode array; and a fixing seat. The fixing seat includes a fixing structure. The fixing seat is mounted on the stretchable covering body through the fixing structure. The fixing seat further includes a hollow chamber for accommodating conductive medium or conductive medium preform. The electrode array includes plug ends, a plurality of electrode sensing units and a plurality of conductive traces. One end of the conductive trace is connected with the electrode sensing unit, and another end of the conductive trace is connected with the plug end. The electrode sensing unit is connected with the fixing seat.

### TECHNICAL FIELD

The present disclosure relates to an electroencephalogram signal recording and acquisition device, and in particular to an electroencephalogram electrode cap.

### BACKGROUND

Electroencephalogram (EEG) is a common bioelectrical signal, which is widely used to obtain neurophysiological information via changes in the electrical signals in brain obtained by measuring the characteristics of the neural potential emitted by the brain. With advancement of medical science and engineering technology, EEG-based electrophysiological monitoring is also rapidly developing in clinical diagnosis and treatment. In recent years, emerging EEG-based clinical applications have become more popular.

EEG recording electrodes are one of key technologies for EEG applications. In the related art, there are mainly two types of EEG recording electrodes, including: an electroencephalogram electrode cap and disk-shaped electrodes. The first type is an electroencephalogram electrode cap, in which, according to the international EEG positioning system, a lot of electrode holders are mounted on an electrode cap, and then electrode sheets with wires soldered thereon are fixed to the electrode holders, and then the wires are arranged on a cap body. The whole manufacturing process is very complicated, and usually includes multiple steps such as pressing electrode materials (e.g. Ag/AgCl fine powder) into electrode sheet, welding wire on electrode sheet, encapsulating solder joints with epoxy resin, and arranging wire on the cap body. Many of the above processes are time consuming and difficult for scale production. In addition, due to clinical requirements for high quality EEG signals, it is usually necessary to pretreat the skin and inject conductive gel to reduce electrode-skin impedance. Due to the large number of electrode channels, it takes a lot of time for medical staff to prepare before the measurement. The preparation time is usually 20-30 minutes. It need more time for electrode setup when it comes to children or patients who do not cooperate. Further, it is often necessary to configure different types of EEG electrode caps (large, medium and small sizes), to meet recording requirements for subjects or patients of different head circumference sizes.

The other type is to employ disc electrodes in a way of fixing disk-shaped electrodes one by one to specified positions of the scalp via viscous conductive paste. Compared with the EEG electrode cap, each disk-shaped electrode is required to be separately positioned, and thus the positioning is relatively inconvenient. Further, recording signals are easily interrupted due to peeling off of the conductive paste during the recording process.

Therefore, EEG acquisition devices in the related art have problems of high price, complicated structure and inconvenient use. There is an urgent need to develop an EEG acquisition device that is simple in assembly, convenient in use, reliable in signal and capable of large-scale production.

### SUMMARY

A main object of the present disclosure is to provide an EEG electrode cap, which can solve the problems of high cost, complicated structure and inconvenient use of the electroencephalogram devices in the related art.

In order to achieve the above object, the present disclosure provides an EEG electrode cap, including: a stretchable covering body; an electrode array; and, a fixing seat. The fixing seat includes a fixing structure; the fixing seat is mounted on the stretchable covering body through the fixing structure. The fixing seat further includes a hollow chamber for accommodating conductive medium or conductive medium preform. The electrode array includes plug ends, a plurality of electrode sensing units and a plurality of conductive traces; one end of the conductive trace is connected with the electrode sensing unit, and another end of the conductive trace is connected with the plug end; and the electrode sensing unit is connected with the fixing seat.

Optionally, a connection between the electrode sensing unit and the fixing seat is adhesion, a non-detachable clamping or non-detachable plugging.

Optionally, a connection between the electrode sensing unit and the fixing seat is a detachable connection.

Optionally, a clamping slot is defined in the fixing seat; and the electrode sensing unit is detachably engaged in the clamping slot.

Optionally, the clamping slot is defined in at least one side surface of the fixing seat; and the electrode sensing unit is inserted into and fixed within the clamping slot through a socket defined by the clamping slot in the side surface.

Optionally, the fixing seat is made of a flexible material.

Optionally, the flexible material is one of silicon rubber, rubber and soft plastic.

Optionally, the fixing seat is made of a rigid material; and a soft supporting body is connected to a lower bottom surface of the fixing seat.

Optionally, a reinforcing plate is disposed between the electrode sensing unit and the fixing seat.

Optionally, a plurality of protruding studs is provided at a lower end surface of the fixing seat.

Optionally, the stretchable covering body is a fabric cap or a fabric strip provided with a mounting hole; and the fixing seat is connected with the stretchable covering body through the mounting hole.

Optionally, the stretchable covering body is a mesh cap; and mesh holes of the mesh cap form the mounting hole.

Optionally, the fixing structure includes a recess defined in a side of the fixing seat; the fixing seat is disposed in the mounting hole; and a portion of the stretchable covering body defining the mounting hole is embedded into and fixed in the recess.

Optionally, the stretchable covering body is a mesh cap, and the fixing seat is further provided with a fixing member; and the fixing member hooks mesh holes of the mesh cap for fixing.

Optionally, the fixing member is a curved hook; or, the fixing member includes a first elastic extension body and a second elastic extension body disposed opposite to each other, and a gap is defined between the first elastic extension body and the second elastic extension body for clamping yarns of the mesh cap.

Optionally, the hollow chamber is filled with a non-viscous conductive medium.

Optionally, the conductive medium preform includes a capsule prefilled with a conductive medium, a liquid absorbing material impregnated with a conductive medium, or a rubber tube filled with a conductive medium.

Optionally, a fixing assembly is provided in the hollow chamber; the fixing assembly includes an upper fastener and a lower fastener; the lower fastener is detachably fixed to the fixing seat; the upper fastener is movably disposed above the lower fastener; the conductive medium preform is in an accommodating space defined by the upper fastener; and a downward movement of the upper fastener relative to the lower fastener compresses the conductive medium preform or presses the conductive medium preform to move downwardly.

Optionally, a piercing structure is provided in hollow chamber of the fixing seat, and the piercing structure is capable of piercing the conductive medium preform to enable the conductive medium to flow out.

Optionally, a frangible structure is provided at one end of the rubber tube.

Optionally, the liquid absorbing material is sealed by a film material.

Optionally, the liquid absorbing material is one or more of foam, absorbent fiber, hydrophilic filter and gel material.

Optionally, a conductive medium is a solution containing sodium chloride or potassium chloride, a conductive glue containing sodium chloride or potassium chloride, or a conductive paste containing sodium chloride or potassium chloride.

Optionally, the electrode array is a flexible electrode array which is fabricated by screen printing or a flexible circuit board process.

### BRIEF DESCRIPTION OF THE DRAWINGS

FIG. 1 is a partial cross-sectional structural view of an EEG electrode cap according to at least one embodiment of the present disclosure;

FIG. 2 is a schematic structural view of an electrode array of an EEG electrode cap according to at least one embodiment of the present disclosure;

FIG. 3 is a partial perspective view of an EEG electrode cap according to at least one embodiment of the present disclosure;

FIG. 4 is a partial cross-sectional structural view of an EEG electrode cap according to at least one embodiment of the present disclosure;

FIG. 5a and FIG. 5b are partial schematic structural views of an EEG electrode cap according to at least one embodiment of the present disclosure;

FIG. 6 is a partial cross-sectional structural view of an EEG electrode cap according to at least one embodiment of the present disclosure;

FIG. 7 is a partial cross-sectional structural view of an EEG electrode cap according to at least one embodiment of the present disclosure;

FIG. 8 is a schematic perspective view of a fixing seat in an EEG electrode cap according to at least one embodiment of the present disclosure;

FIG. 9 is a schematic perspective view of a fixing seat in an EEG electrode cap according to at least one embodiment of the present disclosure;

FIGS. 10a to 10d are schematic plan views of a fixing seat in an EEG electrode cap according to at least one embodiment of the present disclosure;

FIG. 11 is a plan view of parts of an EEG electrode cap according to at least one embodiment of the present disclosure;

FIG. 12 is a partial cross-sectional structural view of an EEG electrode cap according to at least one embodiment of the present disclosure;

FIG. 13 is a partial perspective view of an EEG electrode cap according to at least one embodiment of the present disclosure;

FIG. 14 is a partial cross-sectional structural view of an EEG electrode cap according to at least one embodiment of the present disclosure;

FIG. 15 is a partial cross-sectional structural view of an EEG electrode cap according to at least one embodiment of the present disclosure; and

FIG. 16 is a perspective view of an EEG electrode cap according to at least one embodiment of the present disclosure.

### DETAILED DESCRIPTION

The technical solutions of embodiments of the present disclosure will be described hereinafter in a clear and complete manner in conjunction with the drawings and embodiments. Obviously, the following embodiments are merely a part of, rather than all of, the embodiments of the present disclosure, and based on these embodiments, a person skilled in the art may obtain the other embodiments, which also fall within the scope of the present disclosure.

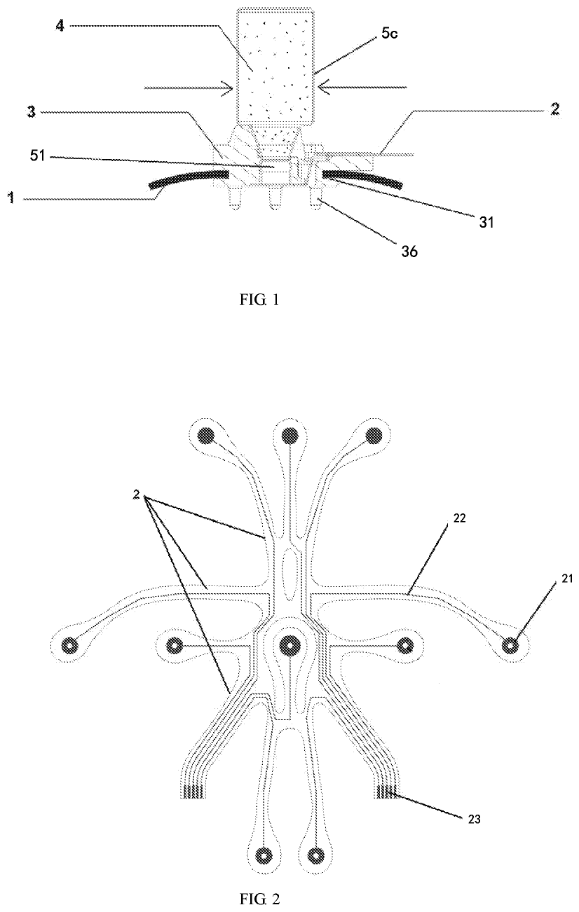

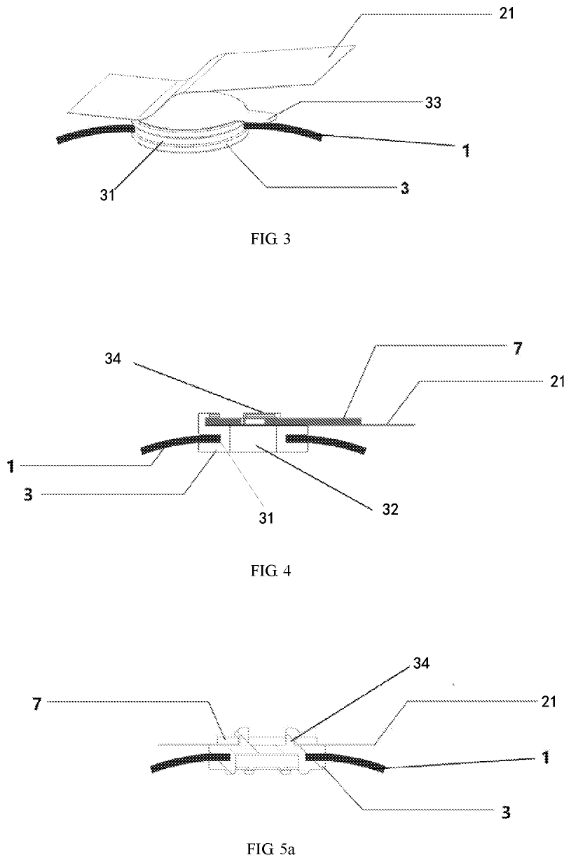

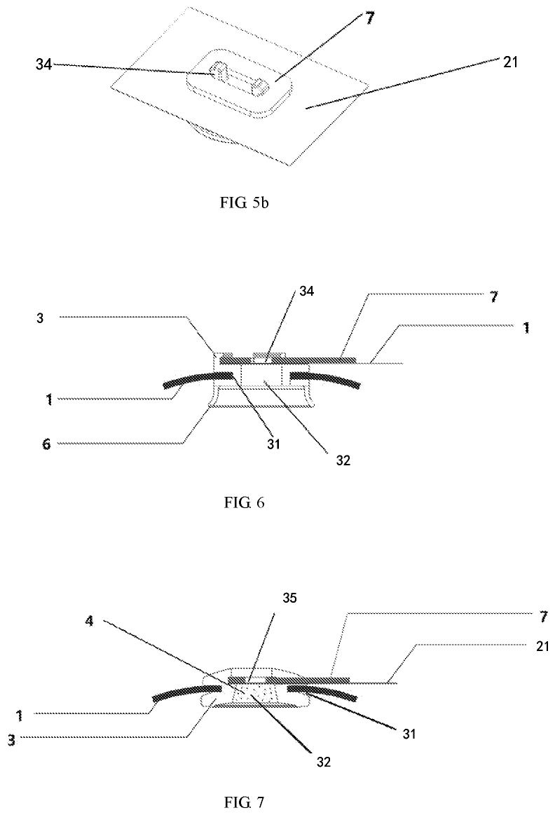

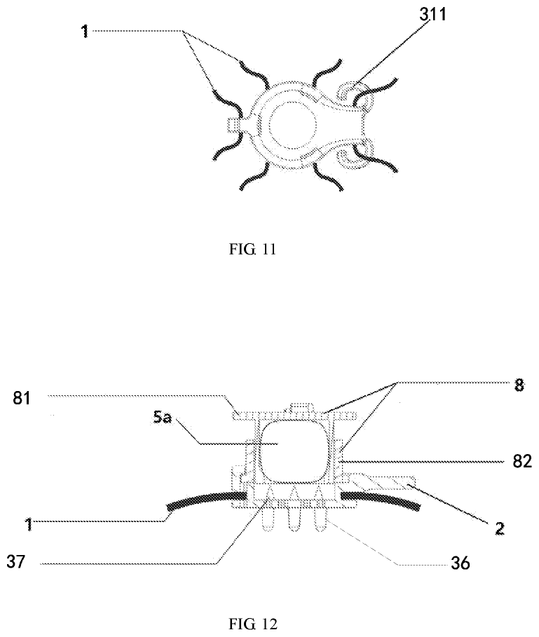

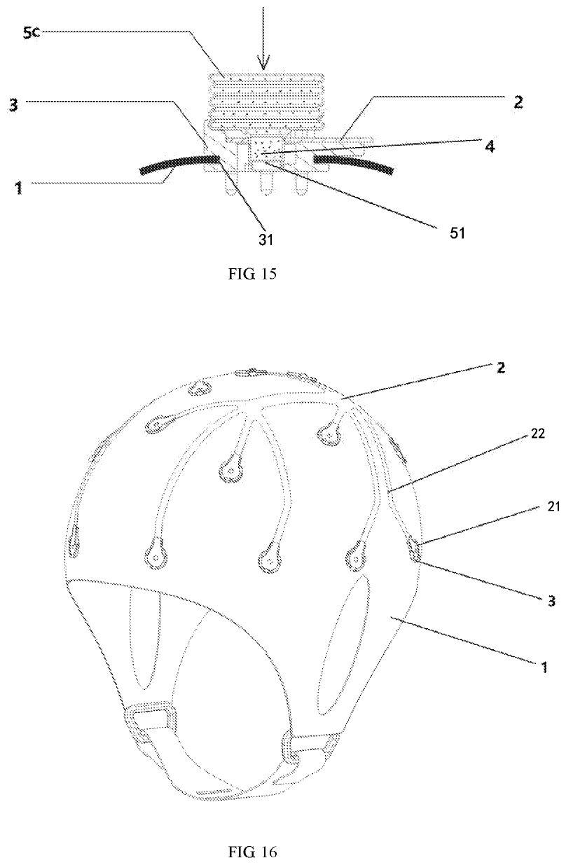

In order to solve the problems of high cost, complicated structure and inconvenient use of the EEG acquisition devices in the related art, according to at least one embodiment of the present disclosure, as shown in FIG. 1, FIG. 3, FIG. 4, FIG. 6, FIG. 7, FIG. 12, FIG. 14, FIG. 15 and FIG. 16, the present disclosure provides an electroencephalogram electrode cap including a stretchable covering body 1, an electrode array 2 and a fixing seat 3. The fixing seat 3 includes a fixing structure 31. The fixing seat 3 can be mounted on the stretchable covering body 1 through the fixing structure 31. The fixing seat 3 further includes a hollow chamber 32 for accommodating conductive medium 4 or conductive medium preform. As shown in FIG. 2 and FIG. 16, the electrode array 2 includes plug ends 23, a plurality of electrode sensing units 21 and a plurality of conductive traces 22. One end of the conductive trace 22 is connected with the electrode sensing unit 21, and the other end is connected with the plug end 23. The electrode sensing unit 21 is connected with the fixing seat 3.

In the electroencephalogram electrode cap provided in the present disclosure, the stretchable covering body 1 has elastic stretchable properties. During use, the stretchable covering body can wrap heads to be inspected, thereby meeting the requirements of EEG measurement at subjects' heads with different head circumferences. In addition, the electrode array 2 for measuring EEG signals may be mounted to the stretchable covering body 1 through the fixing seat 3, and the assembly is very simple and convenient to use.

Further, the stretchable covering body 1 may be a fabric cap or a fabric strip, such as a disposable surgical mesh-cap, or other low-cost mesh caps. Compared with the existing EEG acquisition devices, the EEG electrode cap provided in the present disclosure has the following advantages: the EEG electrode cap is composed of a low-cost electrode array and a stretchable covering body, which greatly reduces the cost of the EEG electrode cap, and it can be used as a disposable consumable, which can effectively avoid cross-infection and meet the low-cost use requirements of EEG electrode caps.

The stretchable covering body 1 provided in the EEG electrode cap can be adapted to subjects or patients of different head circumferences for recording EEG; and positioning assembly is quick and convenient to use.

In addition, quick connection structure between the electrode array 2 and the stretchable covering body 1 can greatly reduce the preparation time of the medical staff before the EEG measurement. Thus, the EEG electrode cap is particularly suitable for recording EEG in complex scenarios such as the intensive care unit and the emergency room.

In at least one embodiment of the present disclosure, as shown in FIG. 3, a connection between the electrode sensing unit 21 of the electrode array 2 and the fixing seat 3 is a non-detachable connection. Optionally, the non-detachable connection between the electrode sensing unit 21 and the fixing seat 3 is adhesion. An adhesive layer 33 is disposed at a top surface of the fixing seat 3, and the electrode sensing unit 21 is adhered to the adhesive layer 33 on the fixing seat 3.

Optionally, a protective film is pre-adhered on the adhesive layer 33 of the fixing seat 3. When used, the protective film on the adhesive layer 33 of the fixing seat 3 is peeled off, and then the electrode sensing unit 21 of the electrode array 2 can be directly adhered to the fixing seat 3, thereby realizing quick and convenient positioning of recording sites. It should be noted that the non-detachable connection between the electrode sensing unit 21 and the fixing seat 3 is not limited to adhesion, and the non-detachable connection may be non-detachable clamping or non-detachable plugging. In order to enhance reliability of connection between the electrode sensing unit 21 of the electrode array 2 and the fixing seat 3, optionally, a reinforcing plate 7 may be further disposed between the electrode sensing unit 21 and the fixing seat 3, as shown in FIGS. 4 to 7.

Specifically, the reinforcing plate 7 is disposed on a surface of the electrode sensing unit 21 distal to the head to be inspected. The reinforcing plate is combined with the electrode sensing unit 21 and then is connected with the fixing seat 3.

Optionally, in some embodiments of the present disclosure, as shown in FIG. 4, FIG. 5a and FIG. 5b, the connection between the electrode sensing unit 21 and the fixing seat 3 is a detachable connection. In one embodiment, the detachable connection is a clamping connection. At least one clamping slot 34 is defined in the fixing seat 3, and the electrode sensing unit 21 of the electrode array 2 is engaged in the clamping slot 34. As shown in FIG. 4, the clamping slot 34 is defined in at least one side surface of the fixing seat 3. The electrode sensing unit 21 of the electrode array 2 is inserted into and fixed within the clamping slot 34 through a socket defined by the clamping slot 34 in the side surface. Optionally, the clamping slot 34 extends through at least two side surfaces of the fixing seat 3 to facilitate installation of the electrode sensing unit 21 in the fixing seat 3 by means of plugging. If necessary, the reinforcing plate 7 may be provided between the electrode sensing unit 21 and the fixing seat 3, thereby make the connection more secure via the increased thickness.

Optionally, in some embodiments of the present disclosure, as shown in FIG. 5a and FIG. 5b, different from the above embodiment, the electrode sensing unit 21 is provided with an opening or a groove, and the clamping slot 34 may be a notch defined in the fixing seat 3 and facing outside. One portion of the fixing seat 3, which defines the clamping slot 34, extends through the opening or groove of the electrode sensing unit 21, so that the electrode sensing unit 21 engages with the clamping slot 34 and is fixed therein. Optionally, the reinforcing plate 7 is provided on a surface of the electrode sensing unit 21 distal to the head to be inspected. The reinforcing plate 7 is provided with an opening or a groove which is corresponding to the opening or groove of the electrode sensing unit 21. When the reinforcing plate 7 and the electrode sensing unit 21 are stacked on each other, the reinforcing plate 7 is engaged with and fixed by the clamping slot 34, thereby securing the electrode sensing unit 21 into the clamping slot 34.

Optionally, in some embodiments of the present disclosure, another example of the detachable connection between the electrode sensing unit 21 and the fixing seat 3 are as shown in FIG. 7. A socket 35 is defined in at least one side of the clamping slot in the fixing seat 3. The electrode sensing unit 21 of the electrode array 2 is inserted into the socket 35 and then fixed. Optionally, the fixing seat 3 is made of a flexible material. The flexible material is one of silicone rubber, rubber and soft plastic. When the fixing seat 3 is made of a flexible material, the EEG electrode cap is comfortable to wear, does not leave an indentation for long-term wearing, and is particularly suitable for sleep monitoring and long-term EEG recording. It should be appreciated that when the fixing seat 3 is made of a rigid material, an equivalent embodiment provides a more reliable plug-in fixation as compared with the flexible material.

Optionally, in some embodiments of the present disclosure, as shown in FIG. 6, in any example of the connection between the sensing unit 21 and the fixing seat 3, optionally, the fixing seat 3 is made of a rigid material. A soft supporting body 6 is connected to a lower bottom surface of the fixing seat 3. Optionally, the flexible supporting body 6 has a flared shape. The presence of the soft supporting body 6 not only increase a contact area between the conductive medium (or a conductive medium flowing out from the conductive medium preform) and the scalp, but also provides comfortable physical contact with the skin, thereby improving the wearing comfort.

Optionally, in some embodiments of the present disclosure, as shown in FIG. 1, FIG. 3, FIG. 4, FIG. 6, FIG. 7, FIG. 12, FIG. 14 and FIG. 15, on the basis of the above implementation structure, mounting holes are pre-defined in the stretchable covering body 1. The fixing structure 31 may be recesses defined in periphery of the fixing seat 3. A portion of the stretchable covering body 1 around the mounting hole is embedded into and fixed in the recess.

Optionally, in some embodiments of the present disclosure, as shown in FIG. 1, FIG. 9 and FIG. 12, a plurality of protruding studs 36 is provided in a lower end surface of the fixing seat 3. When the EEG electrode cap is used to measure EEG signals, the presence of the protruding studs 36 can prevent the fixing seat 3 from sliding on the hair, and can also bring the conductive medium 4 into the hair via friction between the studs 36 and the scalp when the studs 36 are rotated, thereby quickly reducing electrode-skin impedance. Experiments have shown that the electrode-skin impedance can be reduced to less than 5 kΩ by a few simple rotations.

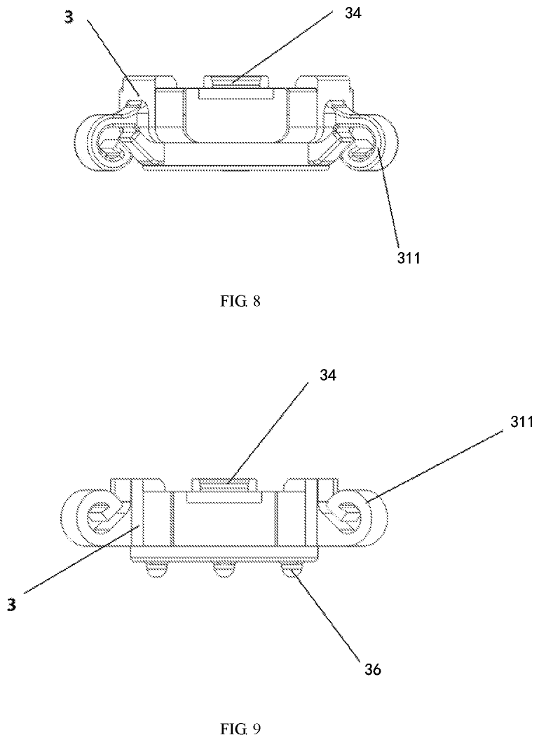

Optionally, in some embodiments of the present disclosure, as shown in FIG. 8 to FIG. 11, the fixing seat 3 is further provided with a fixing member. When the stretchable covering body 1 is a mesh cap, the fixing member can hook yarns of the mesh cap for fixing, the operation is quick and convenient, and the hooked yarns can be prevented from slipping out. As shown in FIG. 8 and FIG. 9, optionally, the fixing member may be formed as a hook 311 that is bent downwardly or/and upwardly to hook the yarns of the mesh cap. It is preferably that the fixing member is an inward-curved hook facing downwardly, which is more convenient and easier to hook the yarns of the mesh cap for fixing.

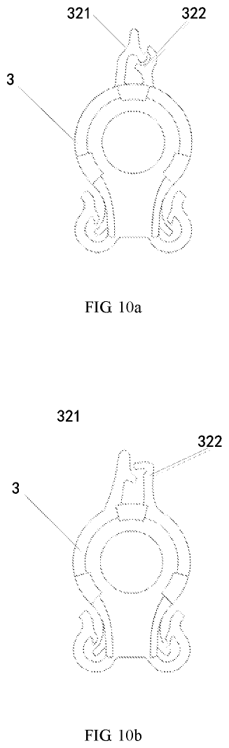

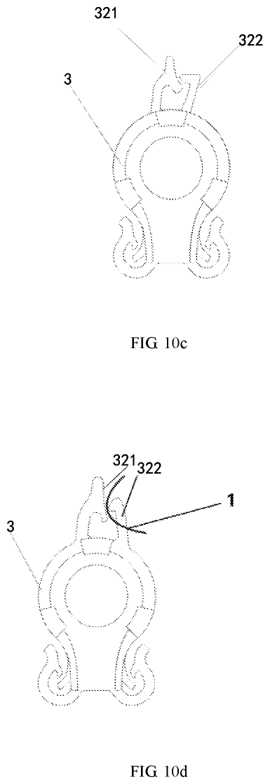

Optionally, in some embodiments of the present disclosure, as shown in FIG. 10a to FIG. 10d, the fixing member disposed on the fixing seat 3 includes a first elastic extension body 321 and a second elastic extension body 322 disposed opposite to each other. A gap is defined between the first elastic extension body 321 and the second elastic extension body 322 for clamping the yarns of the mesh cap. Optionally, a barb is formed on each of the first elastic extension body 321 and the second elastic extension body 322, to prevent the yarns from slipping out. The fixing member of such structure have four variant structures thereof as shown in FIG. 10a to FIG. 10d. The yarns of the stretchable covering body 1 may slide in the fixing member through the gap, and may be prevented from sliding out by a slide-out prevention structure such as the barb, thereby achieving quick fixation of the fixing seat.

Optionally, in some embodiments of the present disclosure, on the basis of the above embodiments, as shown in FIG. 4, FIG. 6 and FIG. 7, the hollow chamber 32 of the fixing seat 3 is filled with a non-viscous conductive medium such as conductive gels, physiological saline or other electrolyte fluids. When the EEG electrode cap is used, the non-viscous conductive medium in the hollow chamber 32 can be permeable from the hollow chamber 32. The non-viscous conductive medium has better fluidity and is more likely to penetrate through dense hair and then permeate into the scalp, thereby achieving lower electrode-skin impedance. In addition, the conductive medium remaining on the head after recording is easier to clean. The viscous conductive paste can serve to secure the fixing seat, even without additional supporter, but the conductive paste is not easy to penetrate through the hair, and the viscous conductive medium (such as conductive paste) adhered to the hair is difficult to clean.

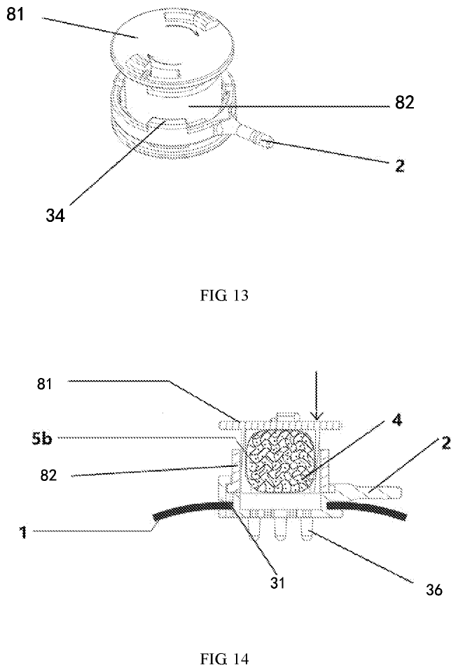

Optionally, in some embodiments of the present disclosure, as shown in FIG. 12 and FIG. 13, the conductive medium preform disposed in the chamber 32 of the fixing seat 3 may be a capsule 5a prefilled with a conductive medium. A piercing structure 37 is provided in the fixing seat 3. Optionally, the piercing structure 37 may be a needle-like structure. By providing the conductive medium in the capsule 5a, it is not necessary to inject the conductive gel when using, but the capsule is pressed to be pierced to cause the conductive gel to flow to the scalp. Thus, the preparation time has been shorten, electrode setup is more convenient and quick. So, the EEG cap is particularly suitable for recording EEG in practical application scenarios such as an emergency department. Optionally, the capsule 5a is fixed by a fixing assembly 8. The fixing assembly 8 includes an upper fastener 81 and a lower fastener 82. The lower fastener 82 is detachably fixed to the fixing seat 3. The upper fastener 81 is capable of pressing the capsule 5a downwardly when the upper fastener is rotated relative to the lower fastener 82. Meanwhile, the piercing structure 37 disposed under the capsule 5a, can pierce a capsule shell and release the conductive medium 4 within the capsule. After the conductive medium 4 in the capsule 5a is completely released, the fixing assembly 8 can be taken out and discarded. Thus, the EEG electrode cap is convenient for the subject to lie down for measurement, and is very suitable for long-term sleep EEG recording.

Specifically, as shown in FIG. 12 and FIG. 13, the upper fastener 81 may be rotatably disposed above the lower fastener 82, and the capsule 5a is located in an accommodating space defined by the upper fastener 81. The piercing structure 37 is located below the capsule 5a. The movement of the upper fastener 81 relative to the lower fastener 82 presses the capsule 5a downwardly, and the piercing structure 37 pierces the capsule to release the conductive medium 4 in the capsule.

Optionally, the conductive medium 4 is a solution containing sodium chloride or potassium chloride, or a conductive glue containing sodium chloride or potassium chloride, or a conductive paste containing sodium chloride or potassium chloride. In another embodiments, the conductive medium contains sodium chloride and potassium chloride. If necessary, surfactants also added in the conductive medium to further reduce the electrode-skin impedance. Preferably, the conductive gel containing sodium chloride or potassium chloride is selected, since it is moderately thick, easy to penetrate through the hair and then be released to the scalp, not easy to dry, capable of maintaining the stability of the electrode-skin impedance, and suitable for sleep or long-term EEG recording. The solution containing sodium chloride or potassium chloride has the advantage of rapidly reducing the electrode-skin impedance, and is particularly suitable for short-term EEG recording. However, the solution is easily evaporated and dried, which causes an increase in the electrode-skin impedance and poor signal quality. Thus, the solution is not suitable for sleep or long-term EEG recordings.

Optionally, in some embodiments of the present disclosure, as shown in FIG. 14, the conductive medium preform is a liquid absorbing material 5b impregnated with a conductive medium, and is fixed by the fixing assembly 8. The fixing assembly 8 includes an upper fastener 81 and a lower fastener 82. The lower fastener 82 is detachably fixed to the fixing seat 3. The liquid absorbing material 5b is located in an accommodating space defined by the upper fastener 81. The upper fastener 81 compresses the liquid absorbing material downwardly in a manner of thread rotation or needle advancement, thereby to release the conductive medium 4 in the liquid absorbing material 5b. On basis of such structure, optionally, the liquid absorbing material 5b may be sealed by a film material, and a piercing structure may be disposed in the hollow chamber of the fixing seat 3 and the piercing structure is capable of piercing through the conductive medium preform to enable the conductive medium to flow out. The piercing structure may be disposed under the liquid absorbing material 5b. When the upper fastener 81 presses the liquid absorbing material 5b sealed by the film material to move downwardly in a manner of thread rotation or needle advancement, the piercing structure can pierce through the film material, and thus the conductive medium flows out.

Optionally, the liquid absorbing material 5b is one or more of foam, absorbent fiber, hydrophilic filter and hydrogel material. The conductive medium 4 may be a solution containing sodium chloride or potassium chloride, a conductive gel containing sodium chloride or potassium chloride, or a conductive paste containing sodium chloride or potassium chloride. A solution containing sodium chloride or potassium chloride is preferred due to its fast adsorption and release rate.

Optionally, in some embodiments of the present disclosure, as shown in FIG. 1 and FIG. 15, the conductive medium preform is a rubber tube 5c filled with a conductive medium. The rubber tube 5c is easily crushed and broken due to a frangible structure 51 provided at one end thereof, so that the conductive medium 4 can easily flow out. During use, it is only needed to tear off the frangible structure 51 to release the conductive medium, and then a signal transmission pathway can be established quickly. The conductive medium is a solution containing sodium chloride or potassium chloride, or a conductive gel containing sodium chloride or potassium chloride, or a conductive paste containing sodium chloride or potassium chloride. A conductive gel containing sodium chloride or potassium chloride is preferred.

Optionally, in some embodiments of the present disclosure, as shown in FIG. 2, the electrode array 2 is formed by a screen-printing process. The electrode sensing unit 21 is formed by silver paste or silver/silver chloride ink through screen printing. The conductive traces 22 are made by a conductive ink through a screen-printing process. The conductive ink is one or more of a silver ink, a conductive carbon ink, and a carbon-silver composite ink. A substrate of the electrode array 2 is made of a flexible polymer material, preferably polyimide (PI) or polyethylene terephthalate (PET).

Optionally, in some embodiments of the present disclosure, as shown in FIG. 2, the electrode array 2 is fabricated by a flexible circuit board (FPC) process. The conductive lines 22 are a gold-plated copper foil formed by a flexible circuit board (FPC) process. The electrode sensing unit 21 is formed by silver ink or silver/silver chloride ink. The substrate of the flexible electrode array 2 is made of a flexible polymer material, preferably polyimide (PI).

Optionally, in some embodiments of the present disclosure, the stretchable covering body 1 is further provided with a wireless amplifier. The electrode array 2 is directly electrically connected to the wireless amplifier. This is particularly suitable for EEG application in moving scenes such as sport. The electrode array can collect EEG signals, and/or electrocardiograph (ECG) signals, and/or electromyography (EMG) signals, and can be widely used.

Optionally, in some embodiments of the present disclosure, the stretchable covering body 1 is a flexible headband. Mounting holes are formed in the flexible headband in a staggered manner. The electrode array is fixed in the mounting holes. This is particularly suitable for an application scenario with less lead, such as neurofeedback rehabilitation training, brain function assessment of auditory evoked potentials.

Optionally, an opening is defined in the bottom of the fixing seat 3 and in communication with the hollow chamber 32. Through the opening, non-viscous conductive medium or conductive medium of the conductive medium preform in the hollow chamber 32 can flow out. The EEG electrode cap provided in the present disclosure can greatly reduce the cost of the EEG electrode cap and may be as a disposable consumable, by adopting an overall design of a low-cost flexible electrode array, a low-cost elastic mesh cap and a fixing seat capable of quickly connecting the flexible electrode array and the mesh cap. In addition, the EEG electrode cap can effectively avoid cross-infection and save the time of medical staff, and is especially suitable for EEG recording in complex environments such as critical illness and emergency. Further, the fixing seat is provided with the fixing member (such as the hook, an elastic gap assembly), which can quickly fix the fixing seat to mesh holes of the mesh cap or hook the yarns of the mesh cap and prevent the mesh cap from slipping out, thereby achieving quick positioning of the electrode sensing unit. In this embodiment, the electrode sensing unit of the flexible electrode array can be quickly installed by means of adhesion, clamping, plugging, etc., and has the advantages of quick assembly and labor saving in the production process.

In some embodiments of the present disclosure, the presence of the conductive media preform can eliminate the need for the medical staff to inject the conductive medium, thereby saving a large amount of preparation time before the measurement. In addition, the presence of the protruding studs on the bottom of the fixing seat can prevent the fixing seat from sliding on the hair, and can also bring the conductive medium into the hair via simple rotation of the studs 36. Meanwhile, rotation of the studs 36 can rub the scalp, thereby achieving the effect of skin pretreatment. In summary, the EEG electrode cap provided in the present disclosure is quick in positioning and assembly, convenient to use, and suitable for recording EEG of subjects with different head circumferences, and can be used as a disposable consumable for clinical diagnosis.

The above are merely the preferred embodiments of the present disclosure. It should be noted that, a person skilled in the art may make improvements and modifications without departing from the principle of the present disclosure, and these improvements and modifications shall also fall within the scope of the present disclosure.

1-24. (canceled)

25. An electroencephalogram electrode cap, comprising:
a stretchable covering body;
an electrode array; and
a fixing seat,
wherein the fixing seat includes a fixing structure; the fixing seat is mounted on the stretchable covering body through the fixing structure; the fixing seat further includes a hollow chamber for accommodating conductive medium or conductive medium preform; the electrode array includes plug ends, a plurality of electrode sensing units and a plurality of conductive traces; one end of the conductive trace is connected with the electrode sensing unit, and another end of the conductive trace is connected with the plug end; and the electrode sensing unit is connected with the fixing seat.

26. The electroencephalogram electrode cap according to claim 25, wherein a connection between the electrode sensing unit and the fixing seat is adhesion, a non-detachable clamping or non-detachable plugging; or a connection between the electrode sensing unit and the fixing seat is a detachable connection.

27. The electroencephalogram electrode cap according to claim 26, wherein a clamping slot is defined in the fixing seat; and the electrode sensing unit is detachably engaged in the clamping slot.

28. The electroencephalogram electrode cap according to claim 27, wherein the clamping slot is defined in at least one side surface of the fixing seat; and the electrode sensing unit is inserted into and fixed within the clamping slot through a socket defined by the clamping slot in the side surface.

29. The electroencephalogram electrode cap according to claim 28, wherein the fixing seat is made of a flexible material; and the flexible material is one of silicone rubber and soft plastic.

30. The electroencephalogram electrode cap according to claim 26, wherein the fixing seat is made of a rigid material; and a soft supporting body is connected to a lower bottom surface of the fixing seat.

31. The electroencephalogram electrode cap according to claim 26, wherein a reinforcing plate is disposed between the electrode sensing unit and the fixing seat.

32. The electroencephalogram electrode cap according to claim 25, wherein a plurality of protruding studs is provided at a lower end surface of the fixing seat.

33. The electroencephalogram electrode cap according to claim 25, wherein the stretchable covering body is a fabric cap or a fabric strip provided with a mounting hole; and the fixing seat is connected with the stretchable covering body through the mounting hole.

34. The electroencephalogram electrode cap according to claim 33, wherein the stretchable covering body is a mesh cap; and mesh holes of the mesh cap form the mounting hole.

35. The electroencephalogram electrode cap according to claim 33, wherein the fixing structure includes a recess defined in a side of the fixing seat; the fixing seat is disposed in the mounting hole; and a portion of the stretchable covering body defining the mounting hole is embedded into and fixed in the recess.

36. The electroencephalogram electrode cap according to claim 34, wherein the stretchable covering body is a mesh cap, and the fixing seat is further provided with a fixing member; and the fixing member hooks mesh holes of the mesh cap for fixing.

37. The electroencephalogram electrode cap according to claim 36, wherein the fixing member is a curved hook; or, the fixing member includes a first elastic extension body and a second elastic extension body disposed opposite to each other, and a gap is defined between the first elastic extension body and the second elastic extension body for clamping yarns of the mesh cap.

38. The electroencephalogram electrode cap according to claim 25, wherein the hollow chamber is filled with a non-viscous conductive medium.

39. The electroencephalogram electrode cap according to claim 25, wherein the conductive medium preform includes a capsule prefilled with a conductive medium, a liquid absorbing material impregnated with a conductive medium, or a rubber tube filled with a conductive medium.

40. The electroencephalogram electrode cap according to claim 39, wherein a fixing assembly is provided in the hollow chamber; the fixing assembly includes an upper fastener and a lower fastener; the lower fastener is detachably fixed to the fixing seat; the upper fastener is movably disposed above the lower fastener; the conductive medium preform is in an accommodating space defined by the upper fastener; and a downward movement of the upper fastener relative to the lower fastener compresses the conductive medium preform or presses the conductive medium preform to move downwardly.

41. The electroencephalogram electrode cap according to claim 39, wherein a piercing structure is provided in hollow chamber of the fixing seat, and the piercing structure is capable of piercing the conductive medium preform to enable the conductive medium to flow out.

42. The electroencephalogram electrode cap according to claim 39, wherein a frangible structure is provided at one end of the rubber tube.

43. The electroencephalogram electrode cap according to claim 39, wherein the liquid absorbing material is sealed by a film material; or the liquid absorbing material is one or more of foam, absorbent fiber, hydrophilic filter and hydrogel material.

44. The electroencephalogram electrode cap according to claim 25, wherein a conductive medium is a solution containing sodium chloride or potassium chloride, a conductive gel containing sodium chloride or potassium chloride, or a conductive paste containing sodium chloride or potassium chloride, wherein the electrode array is a flexible electrode array which is fabricated by screen printing or a flexible circuit board process.
